(19) United States
(12) Patent Application Publication
(10) Pub. No.: US 2005/0096544 A1
(43) Pub. Date: May 5, 2005

(54) Method and apparatus for single transmission Golay coded excitation

(76) Inventors: Xiaohui Hao, Waukesha, WI (US); Richard Yung Chiao, Menomonee Falls, WI (US); Bruno Haider, Ballston Lake, NY (US)

Correspondence Address: MCANDREWS HELD & MALLOY, LTD, 500 WEST MADISON STREET, SUITE 3400, CHICAGO, IL, 60661, US

(21) Appl. No.: 10/697,215

(22) Filed: Oct. 30, 2003

(51) Int. Cl. A61B 8/00

(52) U.S. Cl. 600/447

(57) ABSTRACT

Certain embodiments of the present invention provide a method and system for improved ultrasound imaging using single transmission coded excitation. Certain embodiments include encoding a first ultrasound beam with a first code, transmitting the first ultrasound beam on a first path, encoding a second ultrasound beam with a second code, transmitting the second ultrasound beam on a second path and receiving echo signals from the first and second ultrasound beams. The codes may be complimentary Golay codes or other complimentary codes. The first and second paths may be adjacent scan lines. The method may also include match filtering the echo signals with corresponding matched filters. Match filtered echo signals along adjacent scan lines may be filtered, such as with a lateral averaging filter or other finite impulse response filter. Alternate complementary code transmission helps preserve or improve frame rate while maintaining signal-to-noise ratio improvement and range lobe cancellation.

### RELATED APPLICATIONS

[Not Applicable]

### FEDERALLY SPONSORED RESEARCH OR DEVELOPMENT

[Not Applicable]

### MICROFICHE/COPYRIGHT REFERENCE

[Not Applicable]

### BACKGROUND OF THE INVENTION

The present invention generally relates to ultrasound imaging. In particular, the present invention relates to single transmission of coded excitation signals for ultrasound imaging.

Ultrasound is sound having a frequency that is higher than a normal person may hear. Ultrasound imaging utilizes ultrasound waves or vibrations in the frequency spectrum above normal human hearing, such as the 2.5-10 MHz range. Ultrasound imaging systems transmit ultrasound into a subject, such as a patient, in short bursts. Echoes are reflected back to the system from the subject. Diagnostic images may be produced from the echoes. Ultrasound imaging techniques are similar to those used in sonar and radar.

A medical ultrasound system forms an image by sequentially acquiring echo signals from ultrasound beams transmitted to an object being imaged. An individual beam is formed by transmitting a focused pulse and receiving the echoes over a continuous range of depths. An amplitude of an echo signal decreases significantly for signal reflectors located deeper in the object due to increased signal attenuation of intervening structures, such as intervening tissue layers.

Forming a best possible image at all times for different anatomies and patient types is important to diagnostic imaging systems. Poor image quality may prevent reliable analysis of an image. For example, a decrease in image contrast quality may yield an unreliable image that is not usable clinically. Additionally, the advent of real-time imaging systems has increased the importance of generating clear, high quality images. In conventional ultrasound imaging, a compromise is made between increased penetration of ultrasound signals into an object and resolution of an image formed from resulting echoes. Poor resolution introduces additional noise or interference in an image. Thus, a signal-to-noise ratio (SNR) decreases since the noise, generated by signal amplifiers, for example, may not be reduced to arbitrary low levels.

One way to recover the signal-to-noise ratio is to increase signal energy that is transmitted into the body. A straight-forward way to increase the transmitted energy is to increase the amplitude of the transmitted waveform. However, increasing transmitted amplitude of a signal has limitations related to patient safety, maximum drive levels, and nonlinear distortions. The U.S. Food and Drug Administration (FDA) limits an amplitude of ultrasound pulses used to obtain images of human patients for health and safety reasons. Another straight-forward way is to increase pulse duration such that more energy can be transmitted. However, increasing pulse duration has the undesirable consequence of reducing axial resolution.

A second method to increase the signal energy is to use coded excitation. Coded pulses may be transmitted to overcome a tradeoff between penetration and resolution. A long pulse may be transmitted, and the echoes from an object may be decoded, and filtered to result in a short pulse such that penetration into the object is preserved. One type of code that may be used is a Golay code as disclosed in U.S. Pat. No. 5,984,869, to Richard, Y. Chiao et al., filed on Apr. 20, 1998, and issued on Nov. 16, 1999. Golay codes are complementary codes that are typically used in pairs. Current Golay coded excitation requires two data acquisitions rather than one acquisition. Two acquisitions are done with a first code in a first acquisition and a second code in a second acquisition. Using two codes in two acquisitions doubles a time to acquire ultrasound data. Double transmission of Golay codes degrades a frame rate of an imaging system. Thus, a system with improved resolution and frame rate would be highly desirable.

Golay coded excitation may be used to cancel range sidelobes. Other coded excitation methods create range sidelobes which may appear as artifacts in an ultrasound image. However, Golay coded excitation is rarely used in current systems due to increased transmission and acquisition time. Thus, a system and method that efficiently and effectively use Golay coded excitation would be highly desirable. There is a need for a system and method for Golay coded excitation in ultrasound imaging that eliminates the requirement for double transmission and double acquisition time.

### BRIEF SUMMARY OF THE INVENTION

Certain embodiments of the present invention provide a method and system for improved ultrasound imaging using single transmission coded excitation. Certain embodiments of the method include encoding a first ultrasound beam with a first code, transmitting the first ultrasound beam on a first path, encoding a second ultrasound beam with a second code, transmitting the second ultrasound beam on a second path and receiving echo signals from the first and second ultrasound beams. The codes may be complimentary Golay codes or other complimentary codes. The first and second paths may be adjacent scan lines. The method may also include storing the echo signals. Additionally, the method may include processing the echo signals to produce image data. Echo signals may be filtered, such as with an averaging filter, a match filter, or other filter. The method may also include matching the echo signals with the first and second ultrasound beams.

In another embodiment, a method includes encoding a plurality of signals with a plurality of complimentary codes, transmitting the plurality of signals on a plurality of paths, and processing echo signals produced from the plurality of signals. The codes may be Golay or other codes. The method may also include filtering the echo signals. The method may further include matching the echo signals with the plurality of signals. The method may also include interpolating a plurality of echo signals to form an image data signal. In an embodiment, the first and second ultrasound beams are encoded in first and second transmit focal zones and transmitted on a beam path. The echo signals received from the first and second ultrasound beams are match filtered and averaged between the first and second focal zones.

Certain embodiments provide an improved ultrasound imaging system for transmitted coded ultrasound signals. The system includes a waveform generator for generating waveforms for a plurality of ultrasound signals. The waveform generator encodes said waveforms for a plurality of ultrasound signals. The system also includes a transducer for transmitting ultrasound beams based on said waveforms along a plurality of beam paths. The transducer is also capable of receiving echo signals in response to said ultrasound beams. The system further includes a decoder for converting said echo signals to image data. In an embodiment, the waveform generator encodes said plurality of ultrasound signals with Golay codes. In another embodiment, the waveform generator encodes said plurality of ultrasound signals with complimentary codes. In an embodiment, the decoder further includes a matched filter for filtering the echo signals. The decoder may also include a lateral averaging filter and/or a finite impulse response filter.

The system may also include a beamformer for forming said ultrasound beams from said waveforms. In an embodiment, the beamformer includes a multi-line beamformer. The multi-line beamformer receives a plurality of echo signals in response to an ultrasound signal. The system may additionally include a memory capable of storing at least one of said waveforms and said echo signals. The system may also include a system controller for controlling an imaging mode and parameters of the system. In an embodiment, the waveform generator encodes first and second ultrasound beams with first and second complimentary Golay codes. Then, the transducer transmits the first and second ultrasound beams in first and second focal zones along a same beam path. The decoder match filters echo signals received in response to the first and second ultrasound beams and averages the echo signals between the first and second focal zones.

### BRIEF DESCRIPTION OF SEVERAL VIEWS OF THE DRAWINGS

FIG. 1 illustrates a block diagram an ultrasound imaging system used in accordance with an embodiment of the present invention.

FIG. 2 illustrates a method for ultrasound imaging in accordance with an embodiment of the present invention.

FIG. 3 depicts examples of double and single transmission Golay coding used in accordance with an embodiment of the present invention.

FIG. 4 illustrates a flow diagram for a method for single transmission Golay coding used in accordance with an embodiment of the present invention.

FIG. 5 illustrates transmission of alternating codes on adjacent lines and interpolating received echo signals in accordance with an embodiment of the present invention.

FIG. 6 shows a code sequence for alternating Golay codes between focal zones and scan lines used in accordance with an embodiment of the present invention.

FIG. 7 shows lateral interpolating scheme with alternating codes in a multi-line acquisition used in accordance with an embodiment of the present invention.

FIG. 8 illustrates a system for Golay coded excitation imaging used in accordance with an embodiment of the present invention.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF THE INVENTION

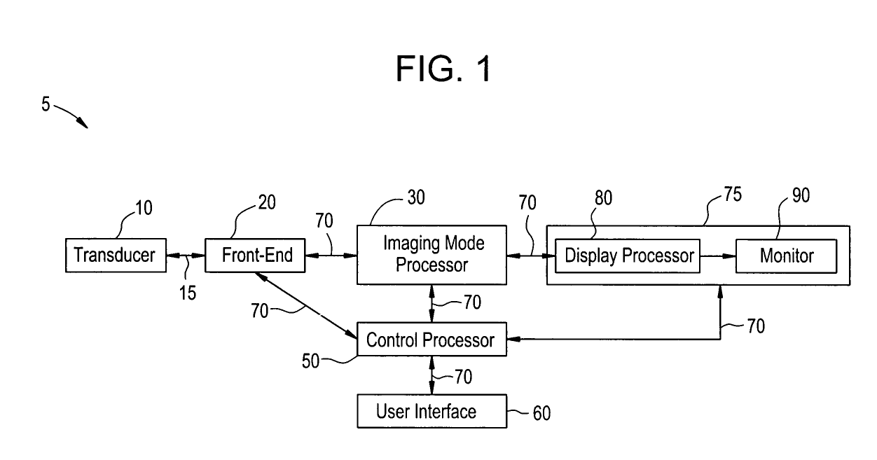

FIG. 1 illustrates a block diagram of an ultrasound imaging system 5 used in accordance with an embodiment of the present invention. The system 5 includes a transducer 10, a front-end 20, an imaging mode processor 30, a user interface 60, a control processor 50, and a display 75. The imaging mode processor 30 and the control processor 50 may be part of a back-end system. The transducer 10 is used to transmit ultrasound waves into a subject by converting electrical analog signals to ultrasonic energy. The transducer 10 also is used to receive ultrasound waves that are backscattered from the subject by converting ultrasonic energy to analog electrical signals. The front-end 20 including a receiver, a transmitter, and a beamformer, is used to create transmitted waveforms, beam patterns, receiver filtering techniques, and demodulation schemes that are used for various imaging modes. The front-end 20 converts digital data to analog data and vice versa. The front-end 20 interfaces with the transducer 10 via an analog interface 15. The front-end 20 interfaces with the imaging mode processor 30 and the control processor 50 via a digital bus 70. The digital bus 70 may include several digital sub-buses. The digital sub-bases may have separate configurations and provide digital data interfaces to various parts of the ultrasound imaging system 5.

The imaging mode processor 30 provides amplitude detection, data compression, and other processing for an imaging mode, such as B-mode imaging, M-mode imaging, BM-mode imaging, harmonic imaging, Doppler imaging, color flow imaging, and/or any other ultrasound imaging mode. The imaging mode processor 30 receives digital signal data from the front-end 20. The imaging mode processor 30 processes the received digital signal data to produce estimated parameter values. The estimated parameter values may be produced using the received digital signal data. The digital signal data may be analyzed in frequency bands centered at the fundamental, harmonics, or sub-harmonics of the transmitted signals to produce the estimated parameter values. The imaging mode processor 30 passes the estimated parameter values to a control processor 50 over the digital bus 70. The imaging mode processor 30 may also pass the estimated parameter values to the display 75 via the digital bus 70.

The display 75 includes a display processor 80 and a monitor 90. The display processor 80 accepts digital parameter values from the imaging mode processor 30 and the control processor 50. The display processor 80 may perform scan-conversion functions, color mapping functions, and tissue/flow arbitration functions, for example. The display processor 80 processes, maps, and formats the digital data for display, converts the digital display data to analog display signals, and passes the analog display signals to the monitor 90. The monitor 90 accepts the analog display signals from the display processor 80 and displays the resultant image. An operator may view the image on the monitor 90.

The user interface 60 allows user commands to be input by the operator to the ultrasound imaging system 5 through the control processor 50. The user interface 60 may include a keyboard, mouse, switches, knobs, buttons, track ball, and/or on screen menus, for example.

The control processor 50 is the central processor of the ultrasound imaging system 5. The control processor 50 interfaces to other components of the ultrasound imaging system 5 using the digital bus 70. The control processor 50 executes various data algorithms and functions for various imaging and diagnostic modes. Digital data and commands may be transmitted and received between the control processor 50 and other components of the ultrasound imaging system 5. In an alternative embodiment, functions performed by the control processor 50 may be performed by multiple processors and/or may be integrated into the imaging mode processor 30 and/or the display processor 80. In another embodiment, the functions of the processors 30, 50, and 80 may be integrated into a single personal computer (PC) backend.

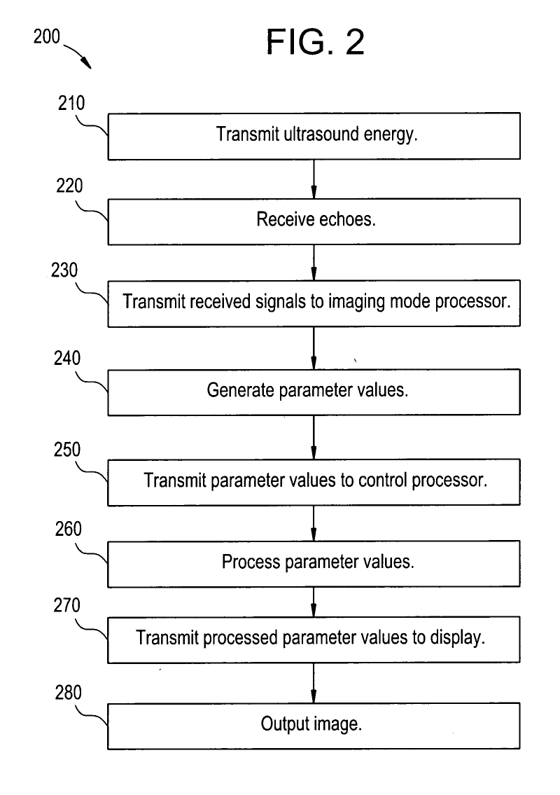

FIG. 2 illustrates a method 200 for ultrasound imaging in accordance with an embodiment of the present invention. First, at step 210, the transducer 10 transmits ultrasound energy into a subject, such as a patient. Then, at step 220, ultrasound energy or echoes backscattered from the subject are received at the transducer 10. Signals are received at the front-end 20 in response to ultrasound waves backscattered from the subject.

Next, at step 230, the received signals are transmitted from the front-end 20 to the imaging mode processor 30 using the digital bus 70. At step 240, the imaging mode processor 30 generates parameter values based on the received signals. Then, at step 250, the parameter values are sent to the control processor 50.

At step 260, the control processor 50 processes the parameter values for use in display, storage, and diagnostics at the display 75. The control processor 50 processes the image data parameter values to reduce artifacts and process resulting image(s).

Next, at step 270, processed parameter values are transmitted to the display 75. The display processor 80 may also process parameter values from a plurality of focal zone images to produce a combined image in conjunction with and/or in addition to the control processor 50.

Finally, at step 280, a diagnostic image is produced and output at the monitor 90. The image may be stored, displayed, printed, and/or further transmitted, for example. The display processor 80 may produce the diagnostic image using the processed parameter values from the digital signal data.

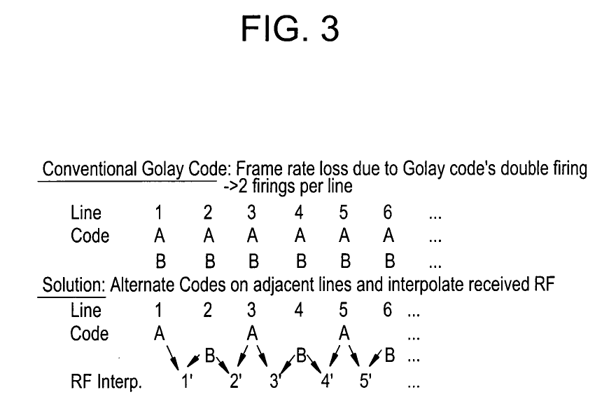

Codes, such as Golay codes, may be used to encode ultrasound signal data to reduce noise in a received echo signal. In an embodiment, Golay codes include two complementary codes “A” and “B”. A double transmission Golay code includes two codes transmitted in sequence. A first code “A” of the code pair is transmitted on a beam path. Then, echoes are received. Next, the received echoes are match filtered using code “A” to form “A*A”, wherein “*” denotes correlation. The correlated echoes are then stored. Next, a second code “B” is transmitted on the beam path. Then, echoes are received. The echoes are match filtered to form “B*B”. The matched filtered signals are added to the filtered echoes of the “A” transmission to form “A*A+B*B”, which results in a final decoded receive pulse. The double transmission and match filtering process is repeated for another ultrasound line. Thus, ultrasound lines are formed by summing matched filtered echoes of “A” and “B” transmissions. An example of double transmission Golay coding is depicted in FIG. 3.

Current Golay coding methods involve transmitting “A” and “B” coded signals along the same path. However, in an embodiment, “A” and “B” codes may be transmitted on spatially adjacent beam paths rather than transmitting codes along the same path. An example of adjacent single transmission Golay coding is shown in FIG. 3. Spatially adjacent complimentary codes may improve frame rate or reduce an impact of coding on transmit and receive frame rate. Use of Golay codes in pairs helps cancel or minimize range sidelobes from received echo signals. Additionally, signal mainlobe energy increases when each code is processed individually and then summed together. Image acquisition time may be decreased and image frame rate may be increased by transmitting one Golay code in any scan direction. Golay codes may be alternated from one scan line to another scan line. However, single codes may introduce range sidelobes in a received echo signal. Sidelobes introduced by single codes may be suppressed by interpolating scan lines that originally had both codes of a Golay pair. In an embodiment, radio frequency (RF) or in-phase and quadrature (IQ) signal data may be used to cancel sidelobes using phase-sensitive interpolation. Scan lines may be interpolated, combined, and/or filtered.

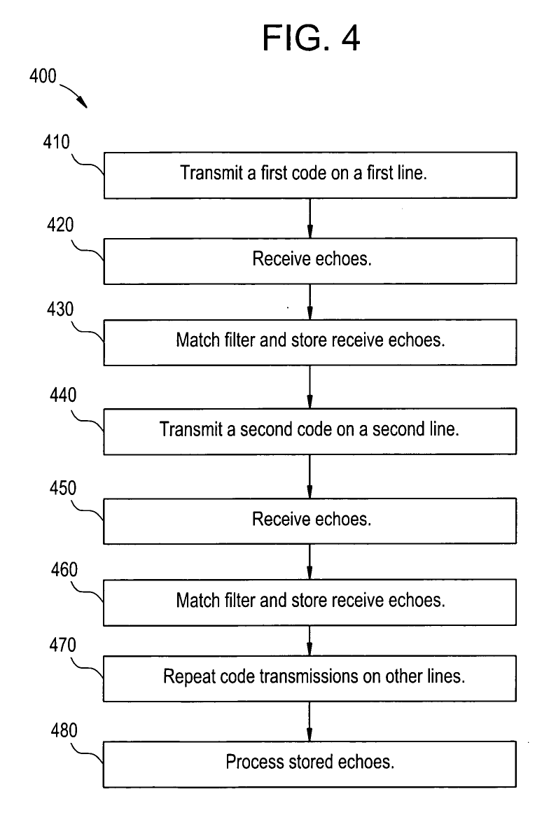

FIG. 4 illustrates a flow diagram for a method 400 for single transmission Golay coding used in accordance with an embodiment of the present invention. First, at step 410, a first code “A” is transmitted on a first transmission line. Then, at step 420, RF echoes are received. Next, at step 430, the received echoes are match filtered and stored. At step 440, a “B” code is transmitted on a second transmission line. Then, at step 450, echoes are received. Next, at step 460, the received echoes are match filtered and stored.

At step 470, the process repeats by transmitting code “A” on odd lines and code “B” on even lines. The echoes are match filtered, and the match filtered echoes are stored. Thus, each ultrasound scan line is formed using a single transmission. Single transmission coding does not increase acquisition time compared to uncoded methods. Then, at step 480, stored RF echo signals are processed by a moving-average lateral filter across ultrasound lines. In an embodiment, the averaging lateral filter has coefficients of [1,1]. An averaging lateral filter with [1,1] coefficients adds two adjacent scan lines. Processed signal data may be used to form an ultrasound image.

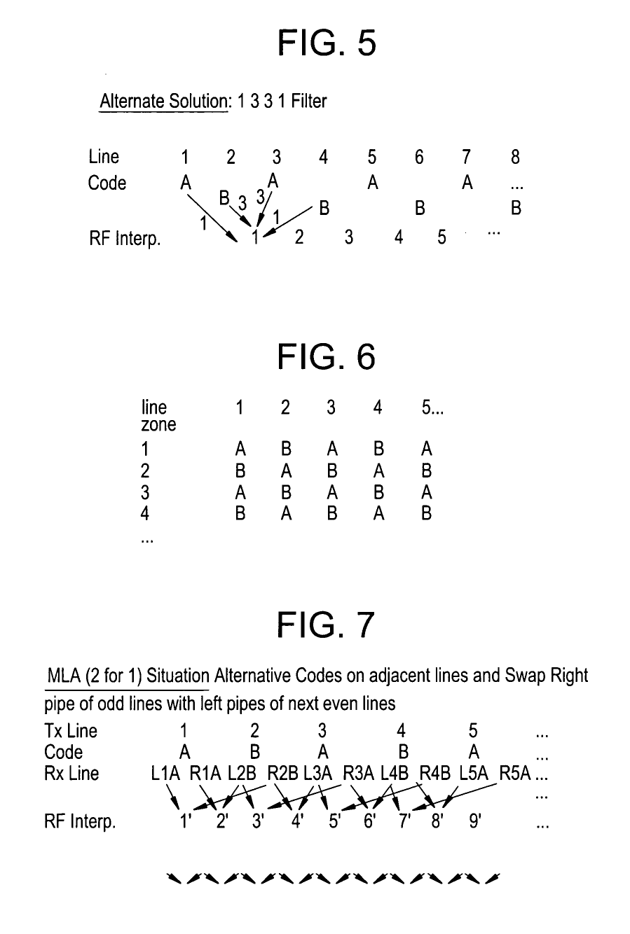

FIG. 5 illustrates transmission of alternating codes on adjacent lines and interpolating received echo signals in accordance with an embodiment of the present invention. The interpolated or averaging lateral filtered lines have a signal-to-noise ratio (SNR) benefit of Golay coded excitation without a loss of frame rate typically associated with Golay codes. Average lateral filtering across ultrasound lines may produce some artifacts in zoomed or magnified images. Artifacts may be reduced or eliminated by applying a more advanced averaging filter.

For example, the [1,1] lateral filter shown in FIG. 5 may be replaced by a higher order lateral filter, such as a [1,3,3,1] filter. The [1,3,3,1] lateral filter reduces artifacts to a level no longer visible in an image. Data acquisition and echo storage may proceed according to the method 400 as described above in relation to FIG. 4. With the [1,3,3,1] filter and other similar lateral filters, the lateral filter operates across ultrasound beams. An ultrasound line is formed from four acquired echo signal lines using the [1,3,3,1] lateral filter. An interpolated line is a sum of two adjacent acquired lines with a weighting factor of three plus a sum of a next adjacent pair of lines with a weighted factor of one, for example.

Other filtering schemes may be derived using standard finite impulse response (FIR) filter or other filter design techniques. Filter designs may optimize a trade-off between image resolution, Golay or other code artifacts, and motion sensitivity, for example. Averaging filters may use even or odd numbers of acquired lines to interpolate. However, interpolation with even and odd number of lines generally produce fewer imaging artifacts. Alternatively, alternating Golay codes “A” and “B” may be applied to subsequent transmit focal zones along the same ultrasound lines. If alternating codes “A” and “B” are applied to subsequent transmit focal zones along the same ultrasound lines, filtering is applied along the focal zones. Each line may be processed independently from adjacent lines. In another embodiment, the Golay codes “A” and “B” may be alternated between transmit focal zones and ultrasound lines as demonstrated in FIG. 6. A filter operation may be two-dimensional, along ultrasound lines, and/or along transmit focal zones, for example.

Alternating Golay codes “A” and “B” between adjacent ultrasound lines may eliminate or reduce doubled acquisition time. An average filtering operation along beams gains SNR benefits from Golay codes. Average filtering along ultrasound beams also reduces or eliminates image artifacts otherwise created by single transmission Golay codes.

Further improvement of frame rate may be achieved using multi-line acquisition (MLA) associated with one transmission. For example, in a two-for-one acquisition, two beams on left and right sides of a transmitted beam path may be received per transmit beam. To combine a two-for-one MLA with Golay “A” and “B” firing, a received left beam of Golay “A” firing is combined with a received right beam of a next Golay “B” firing after matched filtering, and a received right beam of Golay “A” firing is combined with a received left beam of a next Golay “B” firing after matched filtering. Alternate codes on adjacent lines may be combined to be decoded and to avoid beam spatial distortion from multi-line acquisition, as shown in FIG. 7. A [1,1] averaging lateral filter, for example, is used for combination of each two beams. More sophisticated lateral filters may also be applied by carefully select the beams being used.

In an embodiment, the front-end subsystem 20 encodes transmit signals with codes “A” and “B”. The encoded signals are stored in a transmit waveform memory. The front-end 20 transmitter transmits an A-coded beam through the transducer 10 on a beam path. The front-end 20 receiver receives an echo signal through the transducer 10 from the object being imaged. The front-end 20 beamformer forms the received echo into a signal for processing. In an embodiment, one or two beams may be received for each transmitted beam. A user or program may select a frame rate and image quality, for example. For example, one beam is received with a low frame rate and multiple beams may be received at a higher frame rate. In an embodiment, two received beams are positioned to the left and right of the transmitted beam. The received beam(s) are then saved in memory, such as memory in the front-end 20, imaging mode processor 30, control processor 50, and/or other memory, after beamforming of the received echoes by the front-end 20 beamformer.

A B-encoded beam is then transmitted using the transducer 10 to obtain data regarding an object to be imaged. Echoes from the “B” signal are received at the front-end 20, beamformed, and stored in memory, as with signal “A”. The echo signals are then decoded by the front-end 20 using an appropriate decoding filter corresponding to the signal encoding (“A” or “B” for example). The decoded signals are combined to form a decoded composite signal. The imaging mode processor 30 and other system components next perform ultrasound signal and image processing, such as low pass or band pass filtering, scan conversion, etc., as described above. Then, a resulting image is displayed.

For a [1,3,3,1] filter, for example, a memory stores beams corresponding to at least four transmissions, such as “A”, neighbor “B”, neighbor “A”, and neighbor “B”. The beams are transmitted, received, and decoded. After decoding, the decoded signals are passed through the [1,3,3,1] filter to obtain a final pulse for a resulting image.

In an embodiment, the beamformer of the front-end 20 may be a multi-line beamformer. The multi-line beamformer may receive at least two lines for every beam transmitted. A memory in communication with the beamformer is used to store a receive vector for codes “A” and “B”. In the multi-line beamformer, a received echo signal for code “A” may be held in memory until an echo for “B” is received. Then, the echo signals are processed. By receiving two echo signals for every signal transmitted, a frame rate may be doubled using spatially adjacent transmissions.

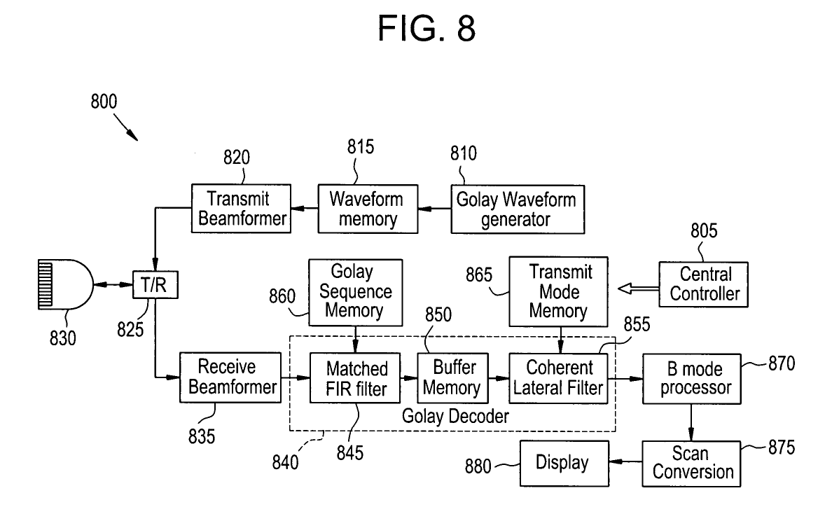

FIG. 8 illustrates a system 800 for Golay coded excitation imaging used in accordance with an embodiment of the present invention. The system 800 may be incorporated into the system 5, may be a separate system, or may be a substitute system. The system 800 includes a central controller 805, a Golay waveform generator 810, a waveform memory 815, a transmit beamformer 820, a transmit/receive switch 825, a transducer 830, a receive beamformer 835, a Golay decoder 840, a Golay sequence memory 860, a transmit mode memory 865, a B-mode processor 870, a scan conversion unit 875, and a display 880. The Golay decoder 840 includes a matched FIR filter 845, a buffer memory 855, and a coherent lateral filter 855. The components of the system 800 may be embodied in software and/or hardware. The components may be separate units and/or may be combined. For example, the waveform memory 815, Golay sequence memory 860, buffer memory 850, and transmit mode memory 865 may be a single memory unit. The components may communicate via wire, wireless, or other connection. The system 800 may utilize Golay codes or other such complimentary codes, for example.

The central controller 805 receives input from a user or a program with an imaging mode and/or other imaging or operational parameters. The imaging mode and parameters are used to generate waveforms and process received signals. Information regarding imaging mode and other parameters may be transmitted to the Golay waveform generator 810. The waveform generator 810 generates one or more waveforms for a transmit signal. The generator 810 encodes the waveform(s) with one or more Golay codes. The generator 810 may also generate delay information for the waveform(s). The waveform(s) and delay information are stored in the waveform memory 815.

The transmit beamformer 820 forms a waveform from the memory 815 into a beam to be transmitted. The beamformer 820 may take into account delay and/or other imaging parameters when forming the transmit beam. The beam is then sent to the transducer 830 through the transmit/receive switch 825. The transmit/receive switch 825 switches between transmit and receive modes in the system 800. The switch 825 allows the transducer 830 to transmit beams and receive echoes. The switch 825 routes signals from the transmit beamformer 820 to the transducer 830 and from the transducer 830 to the receiver beamformer 835.

The transducer 830 transmits the beam(s) into an object. The beam(s) may be transmitted at a certain angle. Echoes backscattered or reflected from structures in the object, such as bones in a patient, are then received at the transducer 830. The transmit/receive switch 825 switches to receive mode and routes the received echoes to the receive beamformer 835. The receive beamformer 835 extracts a received signal from the echo or echoes. The received signal is then sent to the Golay decoder 840.

In the Golay decoder 840, the received signal is first routed to the matched FIR filter 845. The matched filter 845 matches the received signal with the appropriate Golay code used to encode the transmit beam. The Golay sequence memory 860 stores Golay codes for use in matched filtering with the received image data signal. The matched filtered signal is then buffered in the buffer memory 850 for transmission to the lateral filter 855. The coherent lateral filter 855 filters the image data using a filter, such as a [1,1] or [1,3,3,1] moving average filter, for example. The filter 855 decodes the signal and forms an image data signal. The transmit mode memory 865 stores information or parameters related to the selected imaging mode. The imaging mode parameters may be used to determine the filter applied to the matched filtered signal to produce the image data signal.

The B-mode processor 870 receives the image data signal(s) from the Golay decoder 840. The processor 870 processes the image data signal(s) to produce image values or image parameter values. The image data may be analyzed in frequency bands, for example. The image data may also be processed by the processor 870 to remove artifacts and speckle, fine-tune the image, and/or other signal processing, for example. The image/image parameter values are then passed to the scan conversion unit 875. The scan conversion unit 875 prepares the image data for output at the display 880 or other storage. The scan converter 875 provides scan conversion functions, color mapping functions, tissue/flow arbitration functions, formatting functions, and/or other display functions, for example. The display 880 displays the resulting image on a monitor or other medium. The resulting image may also be stored in a memory and/or transmitted.

Thus, certain embodiments of the present invention provide advantages over double transmission Golay coded ultrasound imaging. Certain embodiments provide an SNR gain from coded excitation. Certain embodiments provide reduction or elimination of range sidelobe imaging artifacts. Certain embodiments provide minimal or no loss in frame rate. Certain embodiments may reduce noise in ultrasound, sonar, radar and/or other wave-based systems, for example.

Certain embodiments transmit alternate codes of a Golay pair on adjacent ultrasound transmission lines. Matched filtering is performed on each received echo. Resulting RF or IQ lines are processed with a moving average filter ([1,1] or [1,3,3,1], for example) such that range sidelobes cancel and a mainlobe provides improved SNR. Certain embodiments alternate Golay codes between focal zones and average-filter along focal zones. Certain embodiments alternate Golay codes between focal zones and perform average filtering in, for example, two dimensions. An even or odd number of acquired lines may be used to interpolate. Certain embodiments combine multi-line acquisition with spacially alternating Golay “A” and “B”. A received left beam of Golay “A” firing is combined with a received right beam of a next Golay “B” firing after matched filtering, and a received right beam of Golay “A” firing is combined with a received left beam of a next Golay “B” firing after matched filtering to be decoded and to avoid the beam spatial distortion caused by multi-line acquisition. Multi-line acquisition with alternating Golay codes may further improve frame rate (doubling the frame rate, for example).

Certain embodiments of the present invention reduce or eliminate artifacts through use of alternating Golay codes and an averaging filter. Filtering and interpolation schemes may balance image resolution, artifacts, and motion sensitivity, for example. Certain embodiments cancel or suppress sidelobes and provides an improved signal-to-noise ratio in a mainlobe.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A method for improved coded excitation of ultrasound beams, said method comprising:
encoding a first ultrasound beam with a first code;
transmitting said first ultrasound beam on a first path;
encoding a second ultrasound beam with a second code;
transmitting said second ultrasound beam on a second path; and
receiving echo signals from said first and second ultrasound beams.

2. The method of claim 1, wherein said first and second codes comprise Golay codes.

3. The method of claim 1, wherein said first path and said second path comprise adjacent scan lines.

4. The method of claim 1, further comprising matched filtering of said echo signals.

5. The method of claim 1, further comprising lateral filtering across echo signals from adjacent scan lines.

6. The method of claim 5, wherein said lateral filtering comprises at least one of a lateral averaging filtering and a higher order finite impulse response filtering.

7. The method of claim 1, wherein said first and second ultrasound beams are encoded in first and second transmit focal zones and transmitted on said first path and said second path, wherein said first path equals said second path.

8. The method of claim 7, further comprising:
match filtering said echo signals received from said first and second ultrasound beams; and
averaging between said first and second transmit focal zones.

9. A method for single transmission coded excitation of signals, said method comprising:
encoding a plurality of signals with a plurality of complimentary codes;
transmitting said plurality of signals on a plurality of paths; and
processing echo signals produced from said plurality of signals.

10. The method of claim 9, wherein said codes comprise Golay codes.

11. The method of claim 9, further comprising filtering said echo signals.

12. The method of claim 9, further comprising match filtering said echo signals with corresponding matched filters.

13. The method of claim 9, further comprising lateral filtering a plurality of echo signals to form an image data signal.

14. An improved ultrasound imaging system for transmitted coded ultrasound signals, said system comprising:
a waveform generator for generating waveforms for a plurality of ultrasound signals, wherein said waveform generator encodes said waveforms for a plurality of ultrasound signals;
a transducer for transmitting ultrasound beams based on said waveforms along a plurality of beam paths, said transducer capable of receiving echo signals in response to said ultrasound beams; and
a decoder for converting said echo signals to image data.

15. The system of claim 14, wherein said waveform generator encodes said plurality of ultrasound signals with Golay codes.

16. The system of claim 14, wherein said waveform generator encodes said plurality of ultrasound signals with complimentary codes.

17. The system of claim 14, wherein said decoder further comprises a matched filter for filtering said echo signals.

18. The system of claim 14, wherein said decoder further comprises at least one of a lateral averaging filter and a finite impulse response filter.

19. The system of claim 14, further comprising a beamformer for forming said ultrasound beams from said waveforms.

20. The system of claim 19, wherein said beamformer further comprises a multi-line beamformer, wherein said multi-line beamformer receives a plurality of echo signals in response to an ultrasound signal.

21. The system of claim 14, further comprising a memory capable of storing at least one of said waveforms and said echo signals.

22. The system of claim 14, further comprising a system controller for controlling an imaging mode and parameters of said system.

23. The system of claim 14, wherein said waveform generator encodes first and second ultrasound beams with first and second complimentary Golay codes, wherein said transducer transmits said first and second ultrasound beams in first and second focal zones along a beam path, wherein said decoder match filters echo signals received in response to said first and second ultrasound beams and averages said echo signals between said first and second focal zones.
